(19) United States
(12) Patent Application Publication
(10) Pub. No.: US 2026/0256712 A1
(43) Pub. Date: Sep. 3, 2026

(54) COMPOUNDS FOR INHIBITING GRAM-POSITIVE BACTERIA

(71) Applicants: RUTGERS, THE STATE UNIVERSITY OF NEW JERSEY, New Brunswick, NJ (US); THE UNITED STATES OF AMERICA, AS REPRESENTED BY THE SECRETARY, DEPARTMENT OF HEALTH AND HUMAN SERVIC, Bethesda, MD (US)

(72) Inventors: Wlodek MANDECKI, Newark, NJ (US); Emanuel GOLDMAN, Newark, NJ (US); Maxim CHUDAEV, Newark, NJ (US); Mark James HENDERSON, Rockville, MD (US); Juan Jose MARUGAN, Rockville, MD (US); Wenjuan YE, Rockville, MD (US); Kenneth WILSON, Rockville, MD (US); Dane PARKER, Newark, NJ (US)

(21) Appl. No.: 18/871,571

(22) PCT Filed: Jun. 7, 2023
(86) PCT No.: PCT/US2023/068040

§ 371 Date: Dec. 4, 2024

Related U.S. Application Data
(60) Provisional application No. 63/350,533, filed on Jun. 9, 2022.

(51) Int. Cl. A61K 31/05; A61P 31/04

(52) U.S. Cl. CPC A61K 31/05; A61P 31/04

(57) ABSTRACT

Compounds and methods for their use in inhibiting bacterial protein synthesis in gram-positive bacteria and treating gram-positive bacterial infections in a subject are provided.

This patent application claims the benefit of priority from U.S. Provisional Application Ser. No. 63/350,533, filed Jun. 9, 2022, teachings of which are incorporated herein by reference in their entirety.

This invention was made with government support under grant no. HG004364-01 awarded by the National Institutes of Health. The government has certain rights in this invention.

### FIELD

This disclosure provides compounds for use in inhibiting bacterial protein synthesis in gram-positive bacteria and treating gram-positive bacterial infections in a subject.

### BACKGROUND

There are several gram-positive bacteria that are dangerous pathogens.

For example, Staphylococcus aureus, when present in areas other than its usual carriage in the upper respiratory tract can be difficult to treat, particularly the increasingly ubiquitous methicillin-resistant S. aureus (MRSA). MRSA is presently treated as a last resort with Vancomycin, a drug with many negative side effects. Vancomycin-resistant MRSA has been reported, leaving clinicians with no effective antibiotic treatment.

Anthrax is another disease caused by gram-positive bacteria, Bacillus anthracis. This pathogen achieved notoriety when it was deployed as a bio-terrorism weapon in the United States in 2001.

Several other gram-positive pathogens can also be quite dangerous, including Clostridium, Streptococcus, Enterococcus, Listeria, and Corynebacterium species.

Antibiotic-resistance is an issue for most if not all of these pathogens.

The ribosome is the target for over 50% of existing antibacterial drugs. The majority of existing antibiotics have molecular targets within two ribosomal subunits, 50S and 30S, with thiostrepton, avilamycin, linezolid, streptogramins A and B, chloramphenicol, puromycin, lincoasamides and macrolides targeting 50S and spectinomycin, tetracycline 1 and 2, streptomycin, hygromycin B, paramycin, pactamycin and geneticin targeting 30S.

U.S. Pat. No. 8,323,886 discloses fluorescence methods of detecting a protein-elongation related complex and corollary sequence information.

U.S. Pat. No. 10,174,358 discloses an assay for identification of therapeutics targeting ternary complex formation in protein synthesis.

(R,R)-Tetrahydrochrysene ((R,R)-THC) is known for its biological activity as a non-steroidal, selective estrogen receptor ligand being an agonist at the ERα receptor and an antagonist at the ERβ receptor (see rndsystems with the extension .com/products/rr-thc_1990#product-details of the world wide web. (R,R)-THC is presently used to study estrogen metabolism, with implications for cancer.

There is a need for antibiotics against gram-positive bacteria.

### SUMMARY

An aspect of this disclosure relates to compounds which inhibit ternary complex formation between elongation factor Tu (EF-Tu), transfer ribonucleic acid (tRNA) and guanosine triphosphate (GTP) in gram-positive bacteria.

In one nonlimiting embodiment, the compound comprises Formula I:

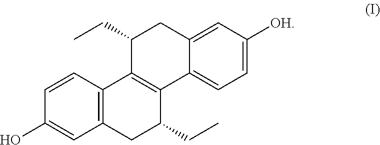 [SMILES: CC[C@@H]1Cc2cc(O)ccc2C2=C1c1ccc(O)cc1C[C@H]2CC]

Another aspect of this disclosure relates to methods for inhibiting bacterial protein synthesis in gram-positive bacteria which comprises contacting the gram-positive bacteria with a compound which inhibits ternary complex formation between EF-Tu, tRNA and GTP in the gram-positive bacteria.

In one nonlimiting embodiment, the method comprises contacting the bacteria with a compound comprising Formula I:

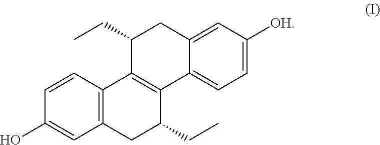 [SMILES: CC[C@@H]1Cc2cc(O)ccc2C2=C1c1ccc(O)cc1C[C@H]2CC]

Another aspect of this disclosure relates to methods for treating a gram-positive bacterial infection in a subject which comprises administering to the subject a composition comprising a compound which inhibits ternary complex formation between EF-Tu, tRNA and GTP in the gram-positive bacteria.

In one nonlimiting embodiment, the method comprises administering to the subject a composition comprising a compound of Formula I:

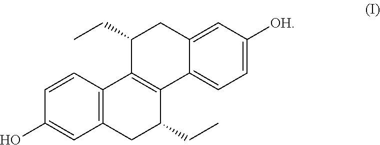 [SMILES: CC[C@@H]1Cc2cc(O)ccc2C2=C1c1ccc(O)cc1C[C@H]2CC]

### BRIEF DESCRIPTION OF THE FIGURES

FIGS. 1A and 1B show the activity scores of compounds from the LOPAC 1280 library in the EF-Tu:tRNA FRET assay with arrow 4 at RR-THC and arrows 1, 2, 3, 5 and 6 at additional compounds with the best rankings (FIG. 1A) and the names of these compounds as well as their gram-positive bacterial inhibitory activity in the Table of FIG. 1B. For these scores, the FRET ratios originating from online (robotic) and offline experiments were subjected to further analysis. First, one score for both online and offline experiment was derived by taking the geometric mean (the square root of the product) of these results where both Max Response values were negative. The scores (Y-axis) of all compounds (X-axis) are plotted in FIG. 1A. Inhibition levels of compound on the FRET assay are indicated on a qualitative scale from 0 to 10, with 10 corresponding to complete inhibition, in the table of FIG. 1B.

FIG. 2 shows a putative generalized structure of compounds active in the EF-Tu:tRNA FRET assay.

FIG. 3 shows a consensus structure of high-scoring compounds active in the EF-Tu:tRNA FRET assay and four nonlimiting examples of specific examples out of a total of 8 in the top-30 as depicted in the Table of FIG. 1B. Numbers indicate the rank of the compound as shown in the Table of FIG. 1B.

FIG. 4 is a bargraph showing results from the in vitro FRET assay wherein the Cy5 acceptor fluorescence intensity significantly diminished in presence of RR-THC.

FIG. 5 is a bargraph showing results of a bacterial growth assay (Bacillus subtilis) in 96 well plate at different concentrations of RR-THC.

FIG. 6 is a photograph showing results of a zone inhibition assay. Bacillus subtilis cells formed a lawn of cells over agar in a Petri dish. Filter #2 contained RR-THC where a clear zone indicative of inhibition of cell growth inside the circle around #2 filter was observed. RR-THC did not inhibit Gram-negative Escherichia coli, indicating that RR-THC is not a non-specific inhibitor of all cells.

FIG. 7 is a bargraph showing ~97% inhibition at 5 μM RR-THC when the cells are grown in test tubes.

FIG. 8 is a bargraph showing results of a drug susceptibility assay determining minimum inhibitory concentration (MIC). As shown, three Staphylococcus aureus strains were highly sensitive to RR-THC including the MRSA strain (methicillin-resistant S. aureus). Gram-negative bacteria and mycobacteria, were not sensitive to RR-THC, confirming the general specificity of the compound.

FIGS. 9A, 9B, and 9C show results of topical administration of RR-THC inhibiting dermonecrosis in mice infected with S. aureus. FIG. 9A is a linegraph comparing dermonecrosis over time in mice treated topically with RR-THC, mupirocin and vehicle controls; FIG. 9B shows dermonecrosis on Day 5 with each point representing a mouse and lines displaying the median; and FIG. 9C shows bacterial burden quantified from punch biopsies with each point representing a mouse and lines displaying the median.

### DETAILED DESCRIPTION

Ternary complex formation between elongation factor Tu (EF-Tu), tRNA and GTP is a crucial step of protein biosynthesis for delivery of amino acids to the growing protein chain by bringing amino acid-charged tRNA into the codon-programmed A-site of the ribosome. Although more than half of all antibiotics target bacterial protein synthesis, there are no antibiotics in clinical use that target this step. The disruption of the EF-Tu:tRNA complex by a small molecule (the antibiotic being sought) will prevent protein synthesis and result in arrest of bacterial growth. EF-Tu is an ideal target because the protein is essential and highly conserved among bacteria. Further, EF-la, the eukaryotic homolog to EF-Tu, as well as mitochondrial EF-Tu, are sufficiently different from the bacterial form such that bacterial inhibitors are unlikely to affect mammalian cells.

This disclosure relates to identification of compounds which inhibit ternary complex formation between EF-Tu, tRNA and GTP in gram-positive bacteria and to methods for use of these compounds in inhibiting bacterial protein synthesis in gram-positive bacteria and as antibiotics in treating gram-positive bacterial infections in a subject.

For the purposes of this disclosure, the term “subject” is intended to include any mammal including, but not limited to, horses, cows, sheep, goats, dogs, cats, primates and humans. In one nonlimiting embodiment, the subject is a human.

By “treat”, “treatment” or “treating” of a bacterial infection as used herein, it is meant to include the inhibition, reduction or prevention of any gram-positive bacterial infection as well as the inhibition, reduction or prevention of symptoms associated with any gram-positive bacterial infection.

Upon formation of functional ternary complex between Cy5-EF-Tu:GTP and Cy3-Phe-tRNAPhe, fluorescence energy transfer (FRET) occurs between Cy3 and Cy5 fluorophores located within Förster radius (~17 Å) when Cy3 is excited at 532 nm, and as a result the complex emits light at 665 nm. Presence of inhibitors of the ternary complex prevents complex from forming, thereby reducing fluorescence intensity at 665 nm.

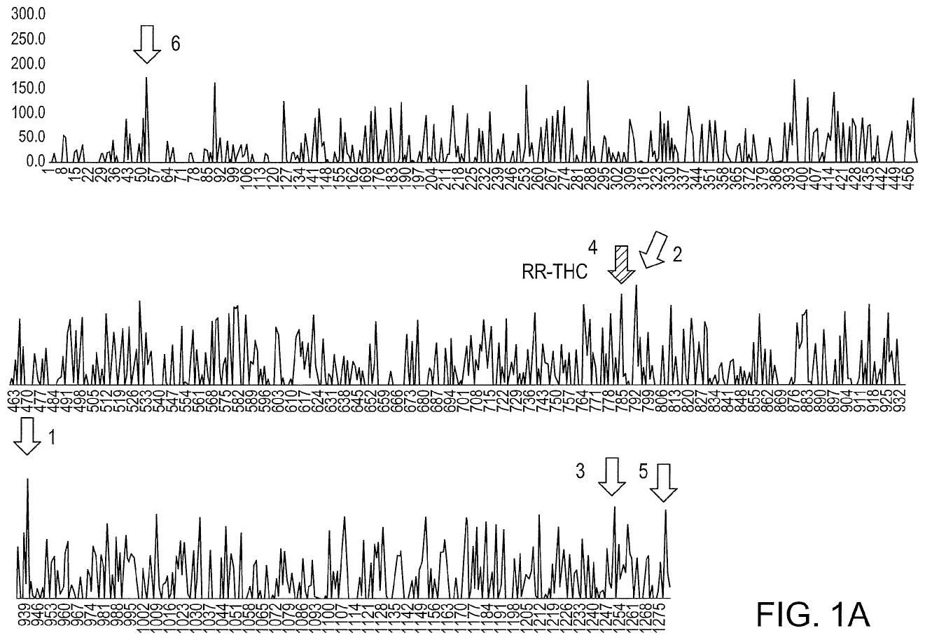

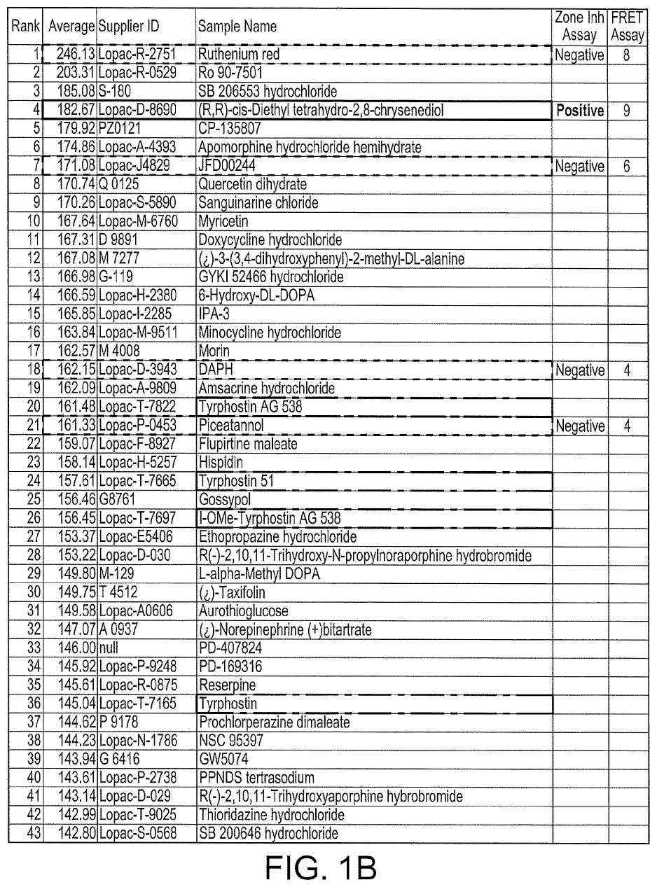

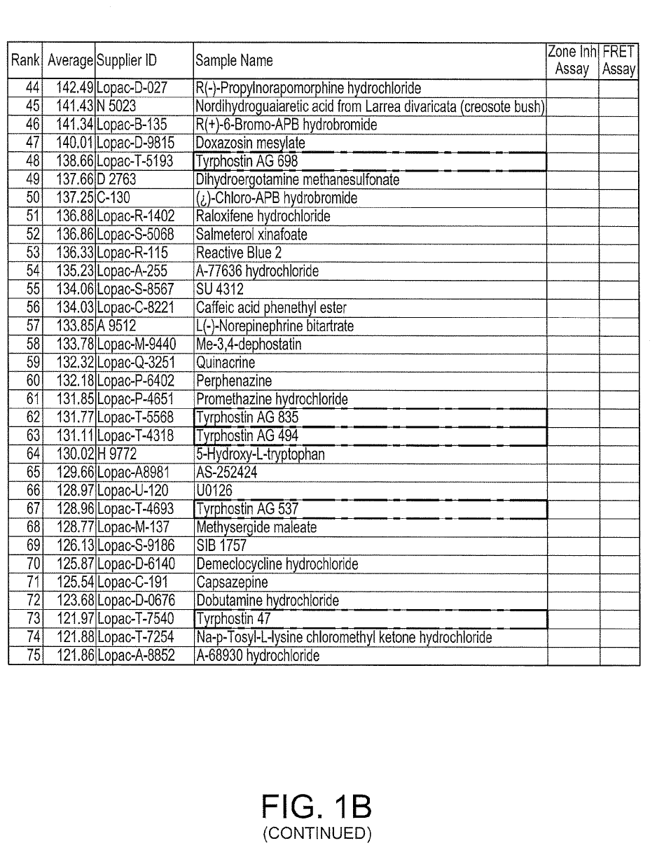

Inhibitors of EF-Tu:tRNA translation were identified using this in vitro FRET system. Quantitative high throughput screening (qHTS) was performed on the LOPAC library containing 1,280 chemicals to identify potential leads for antibiotic development. Results for the 75 top-ranked compounds based upon this in vitro FRET system are depicted in FIGS. 1A and 1B as well as their activity against gram-positive bacteria in vivo using disc-diffusion, microtiter plate and viable count assay.

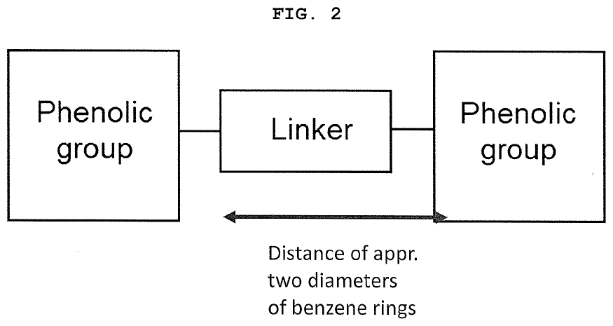

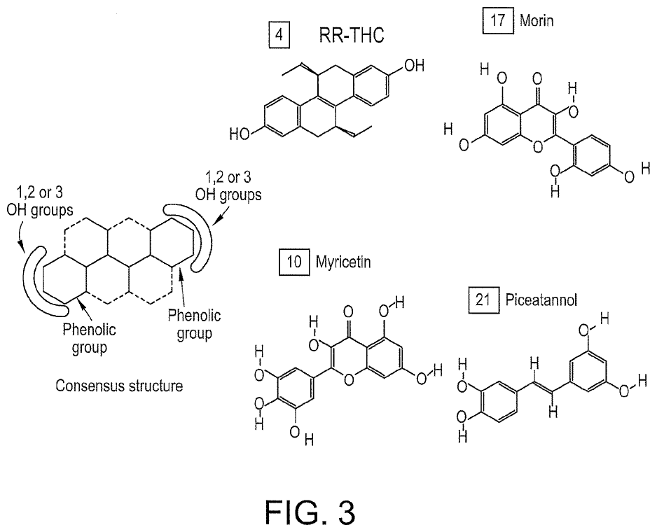

Focusing analysis on thirty chemical compounds with the highest inhibitory activity in the FRET assay revealed a tendency of the compounds to be mono- or bi-phenolic compounds. More specifically, of the top 30 highest ranked compounds, 60% (18 total) have one (6 compounds identified) or two (12 compounds identified) phenolic groups. Within the latter group, the linker length was three atomic bonds for 8 compounds. A putative generalized structure of compounds identified as active in the EF-Tu:tRNA FRET assay is depicted in FIG. 2 while FIG. 3 shows a generalized structure of high-scoring compounds active in the EF-Tu:tRNA FRET assay and four nonlimiting examples of specific compounds out of a total of 8 in the top-30 as depicted in the Table of FIG. 1B. Specific compounds tested fitting the consensus chemical structure include RR-THC, quercetin dihydrate, myricetin, morin, piceatannol, hispidin, and taxifolin.

In addition, tyrphostins were identified as highly represented in the 75 top-ranked compounds. There are 9 tyrphostins in this group, out of a total of 14 tyrphostins in the LOPAC library.

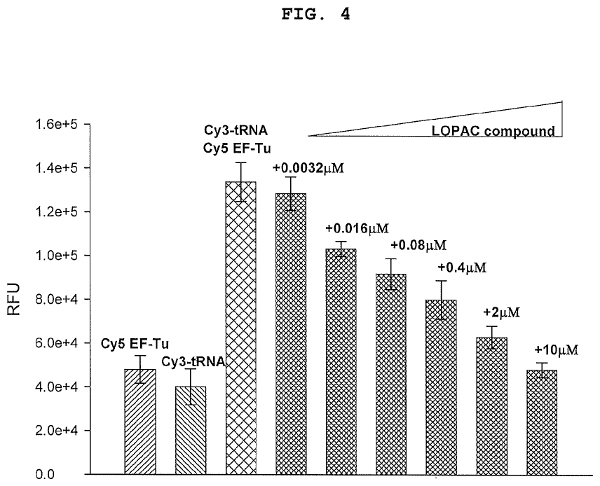

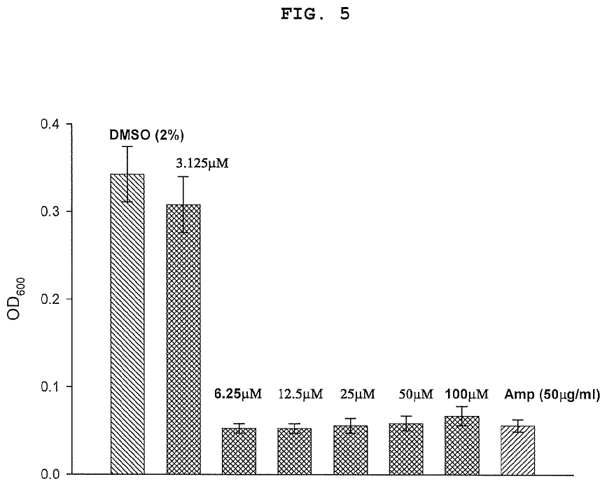

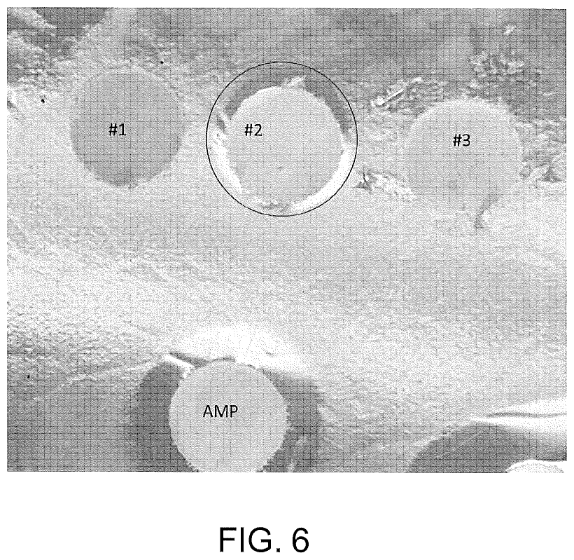

One of the compounds, (R,R)-tetrahydrochrysene (RR-THC), effectively decreased Cy5-EF-Tu acceptor fluorescence emission in the FRET assay (FIG. 4) and also showed activity against gram-positive bacteria in vivo using disc-diffusion, microtiter plate and viable count assay (FIGS. 5-7).

When 10 μM RR-THC was added to the medium in an agar plate and B. subtilis cells were spread over the plate to form colonies, colonies did not form (FIG. 7). Further, as shown in FIG. 7, ~97% inhibition was observed at 5 μM RR-THC when the cells were grown in test tubes. These results suggest that the RR-THC compound is bactericidal rather than bacteriostatic, and that mutations to resistance are rare.

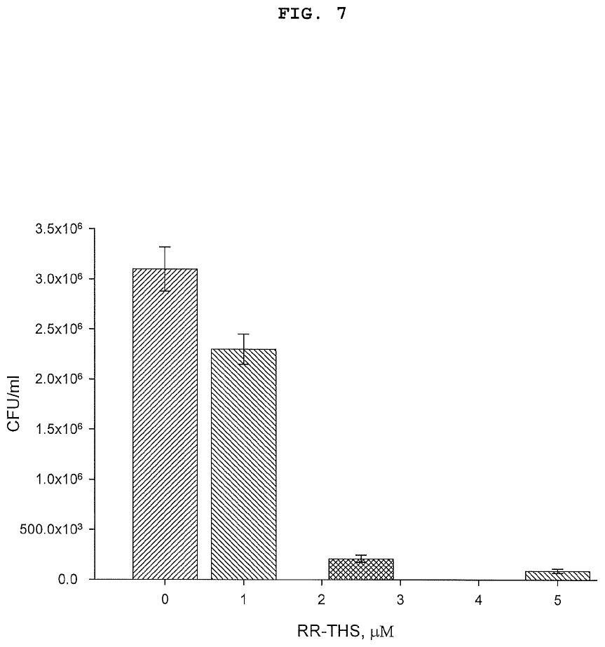

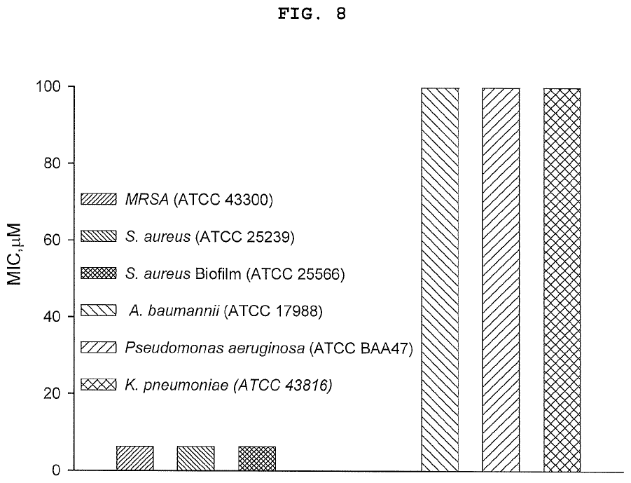

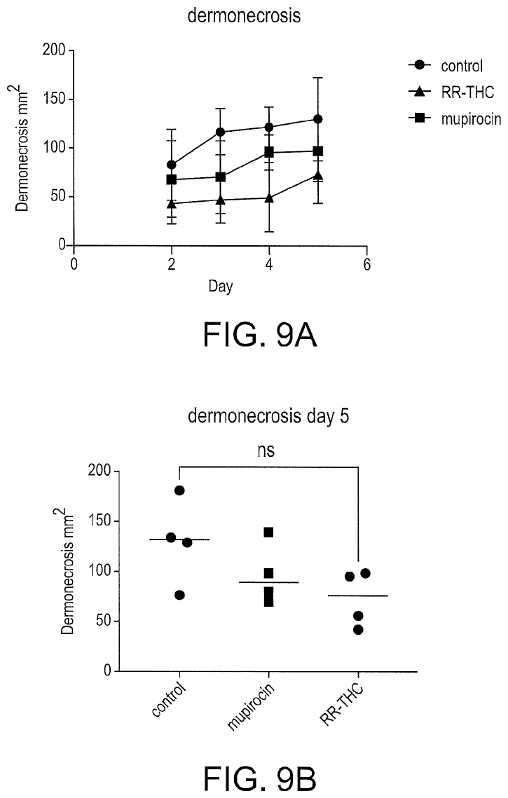

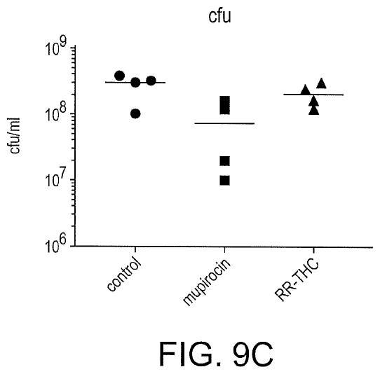

Further, as shown in FIG. 8, three Staphylococcus aureus strains were highly sensitive to RR-THC including the MRSA strain (methicillin-resistant S. aureus), indicating clinical potential of this compound in treating challenging MRSA infections. Gram negative bacteria, and mycobacteria, were not sensitive to RR-THC, confirming the general specificity of the compound. Cytotoxicity against mammalian Vero cells shows a CC 50 value above 50 μM.

A second compound with the consensus chemical structure, piceatannol, was also tested in the zone inhibition test for gram-positive bacteria and was negative (see FIG. 1B). It is expected that testing a larger group of compounds having the consensus structure shown in FIG. 3 will result in identification of additional compounds having antibiotic activity. Further, variations of R,R-THC can now be derived which can penetrate Gram-negative bacteria or mycobacterial species including, but not limited to, the causative agent of tuberculosis).

The accumulation of RR-THC in the liver and a level of toxicity observed upon intraperitoneal administration of 10 mg/ml in mice has led to investigations of topical applications.

Mice were infected subcutaneously with S. aureus applied to their shaved backs while under anesthesia (ketamine/xylazine). Five minutes after inoculation, 10 mg of ointment (0.2 mg of either RR-RHC or mupirocin) was applied to the regions of inoculation. Ointment was applied daily. Regions of dermonecrosis were measured daily. The experiment was stopped after day 4, 24 h prior to euthanasia and processing. The RR-THC treated group showed a trend to reduced dermonecrosis. This is indicative of active infection and is driven by toxin production. Of note, no decrease in the control mupirocin-treated group was observed. However, bacterial counts showed a trend to reduction in the mupirocin group, with no difference apparent in the RR-THC group. Repetition of the experiment showed a statistically significant decrease in dermonecrosis in the RR-THC treatment group compared to controls but not compared to the mupirocin treated group.

Accordingly, a nonlimiting embodiment of this disclosure relates to compositions comprising RR-THC as depicted in Formula I:

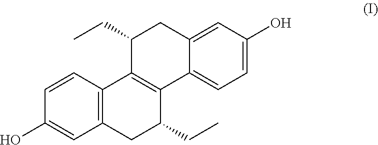 [SMILES: CC[C@@H]1Cc2cc(O)ccc2C2=C1c1ccc(O)cc1C[C@H]2CC]

and methods for use of RR-THC and compositions thereof to inhibit bacterial protein synthesis in gram-positive bacteria and as antibiotics in treating gram-positive bacterial infections in a subject.

In one nonlimiting embodiment, the compositions comprise RR-THC in an amount effective to inhibit bacterial protein synthesis in gram-positive bacteria.

In one nonlimiting embodiment, the compositions comprise RR-THC in an amount effective to treat a gram-positive bacterial infection in a subject.

In addition to RR-THC, compositions of this disclosure may further comprise pharmaceutically acceptable excipients and/or secondary active agents effective against bacteria.

In one nonlimiting embodiment, compositions of this disclosure are formulated for oral administration. For oral administration, RR-THC, together with a conventional adjuvant, carrier, or diluent, alone or in combination with other medications as described herein, may be placed into the form of pharmaceutical compositions and unit dosages thereof in accordance with methods well known to those skilled in the art, and in such form may be employed as solids, such as tablets or filled capsules, or liquids such as solutions, suspensions, emulsions, elixirs, or capsules filled with the same, all for oral use.

In one nonlimiting embodiment, compositions of this disclosure are formulated for topical administration. For topical administration to the epidermis RR-THC alone or in combination with other medications as described herein may be formulated as an ointment, cream or lotion in accordance with methods well known to those skilled in the art. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The following nonlimiting examples are provided to further illustrate the present invention.

### EXAMPLES

### Example 1: Preparation of Reagents for qHTS and Secondary FRET Assays

Cy5-EF-Tu: EF-Tu from E. coli was expressed and labeled with Cy5 as described by Bhatt et al. (Assay Drug Dev Technol. 2018 16(4):212-221) and Chudaev et al. (Protein Eng. Des Sel. February 26 (2013). Sulfonated versions of Cy5 maleimide and Cy3 NHS ester were obtained from GE Healthcare/Amersham or Lumiprobe. Ammonium acetate, magnesium acetate, magnesium chloride, potassium chloride, HEPES, DTT, ATP, GTP, TCEP, L-phenylalanine were obtained from Sigma.

Cy-3-tRNA: tRNA originated from three sources:
(1) Purified preparations of E. coli tRNA^{Phe} purchased from Chemical Block (Moscow, Russia).
(2) tRNA prepared from E. coli cells that overexpress tRNA^{Phe} according to Bhatt et al. (Assay Drug Dev Technol. 2018 16(4):212-221), Chudaev et al. (Protein Eng. Des Sel. February 26 (2013) and Cayama et al. Nucleic Acids Res 2000 28(12): E64.
(3) Commercially available preparation of total tRNA (E. coli MRE 600) from Sigma. After labeling and purification, a mix of Cy3-tRNA^{Phe} and Cy3-tRNA^{Arg} was obtained. The labeling with Cy-3 was performed as described by Bhatt et al. (Assay Drug Dev Technol. 2018 16(4):212-221) and Chudaev et al. (Protein Eng. Des Sel. February 26 (2013).

### Example 2: Secondary FRET Assay

Equipment: EnVision 2104 Multilabel Plate Reader (PerkinElmer) with excitation optical filter 2100-5050 Bodipy TMR FR 531 nm and emission optical filter 2100-5770 Cy5 685 nm.

Reagents: Cy5-EF-Tu and Cy-3-tRNA, were prepared as described in Example 1.

All solutions were prepared fresh in assay buffer consisting of 70 mM HEPES-KOH (pH7.6), 52 mM ammonium acetate, 8 mM magnesium chloride, 30 mM potassium chloride, and 2.6% glycerol. A 200 nM Cy5-labeled EF-Tu working solution was prepared in assay buffer plus 1.4 mM DTT. A 80 nM Cy3-tRNA(Phe) working solution was prepared in assay buffer containing 20 uM Phenylalanine, 2 mM ATP and 7 μg/ml recombinant Phe-tRNA synthetase. Solutions were incubated at 37° C. for 15-25 minutes. 3 uL of the Cy5-EF-Tu (or buffer only control) was dispensed into each well of a black solid 1536-well plate using a Multidrop Combi dispenser (Thermo). Plates were centrifuged for 30 seconds at 650×g (Eppendorf 5804R). 23 nL of compounds were transferred to the wells using a Kalypsis pin-tool and plates were incubated at room temperature protected from light for 15 min. Then, 3 uL of Cy3-tRNA (or buffer only control) was dispensed into each well. FRET was then detected using 1) a ViewLux high-throughput microplate imager (PerkinElmer) equipped with Ex: 525/20 and Em: 671/8 filters; or 2) an Envision Multimode plate reader (PerkinElmer) equipped with Ex: 530/25 and Em: 665 filters. Assay performance, as calculated by Z′ factor as described by Zhang et al (J Biomol Screen, 1999), was favorable for the Envision plate reader.

Factumycin, Kirromycin, and GE2270 were tested as control compounds. Each showed inhibition of FRET signal with an EC₅₀ between 1-5 uM.

Prior to screening, the stability of the reagents and compatibility with HTS was examined by preparing Cy5-EF-Tu and Cy3-tRNA working solutions and incubating them at room temperature over 24 hours. FRET and inhibition by Factumycin and Kirromycin was detectable at each timepoint, supporting stability of the assay, particularly up to 4 hours.

The assay performance with varying incubation times after addition of the Cy3-tRNA component was assessed, using factumycin, kirromycin, and GE2270 as controls. Compound inhibition profiles were similar from 4-12 min, but diminished with a longer incubation of 45 minutes.

Experiments were also performed using a fully automated procedure on a robotic HTS platform. The LOPAC1280 library was selected for testing using a manual assay (“offline”) versus a fully automated procedure (“online”). The control compound kirromycin performed similarly using both procedures.

Results demonstrated substantial agreement for LOPAC testing for both methods (using an Envision plate reader to detect FRET in both cases). Fluorescence values were normalized to DMSO controls (0%) and 40 uM kirromycin (−100%). Compounds that showed large discrepancies between the two methods are shown as small cross hatched bars (FIG. 4). RR-THC was highly active in both methods (FIG. 4). Five compounds, including RR-THC were further analyzed to determine whether they were bona fide EF-Tu:tRNA modulators, or fluorescence artifacts.

### Example 3: Counterscreen Assay Aimed at Excluding Quenching as Mechanism of Inhibition of FRET

Oligonucleotides were purchased from Integrated DNA Technologies:

| (SEQ ID NO: 1) | |
|---|---|
| (1) | CTC TGG GAA CAT CCT/3′Cy5Sp/ |
| (SEQ ID NO: 2) | |
| (2) | /5′Cy3/TT TTT AGG ATG TTC CCA GAG |

In the assay, a 50 nM final concentration of each oligonucleotide was used.

Incubation buffer: 70 mM HEPES-KOH (pH 7.6), 52 mM ammonium acetate, 8 mM magnesium chloride, 30 mM potassium chloride and 2.6% glycerol. A 96-well black, clear bottom microplate was used and read the plate was read using EnVision multilabel plate reader (PerkinElmer) having the same optical filters and parameters as described in Example 2.

Assay: The assay was performed as in Example 2 The presence of RR-THC did not significantly change FRET suggesting that the effect of RR-THC on fluorescence in qHTS is minimal if any (i.e., no quenching).

### Example 4: Zone Inhibition Assay (ZIA)

### Bacterial Strains and Reagents:

B. subtilis 1S-75, E. coli DH5α
DMSO, molecular biology grade from Sigma
LB LENOX and LB MILLER from IBI (ibisci.com)
29 compounds from LOPAC library including RR-THC

### Procedure:

An overnight culture of B. subtilis was diluted 1/1000 in LB Lenox media and grown up to OD₆₀₀=0.1. An 150 μl aliquot of the culture was plated on LB Lenox media Petri dish with 1.5% agar followed by placing of a 5 mm Whatman filter disk containing 3 μl of 10 mM stock of the tested compound (30 nmoles total) and incubated overnight at 30° C. E. coli was incubated at 37° C. in LB Miller media. Commercially available disks containing 100 μg carbenicillin or 50 μg ampicillin (obtained from Pfizer) were used as controls. After the incubation, images of the plates were taken and the size of a halo was qualitatively characterized on a five-element scale: from no halo NA, to one, two, three, four, or five stars, with five stars corresponding to the largest halo.

A total of 32 compounds were tested in the ZIA with compounds sanguinarine chloride (****/****), IPA-3 (**/**), doxycycline (*****/*****), gossypol (**/**), minocycline (*****/****) and RR-THC (***/NA) and controls carbenicillin (****/**) and ampicillin (*****/***) being active in the ZIA. Qualitative results for the B. subtilis/E. coli halos are shown in parenthesis.

### Example 5: Obtaining an Estimate of MIC Based on Bacterial Growth Experiments

### Bacterial Strains and Reagents:

B. subtilis IS-75 and E. coli DH5α
Saline solution (0.9% NaCl)
DMSO molecular biology grade from Sigma
LB LENOX and LB MILLER from IBI (ibisci.com)
RR-THC from TOCRIS (www.tocris.com)

Overnight culture of B. subtilis was diluted 1/1000 in LB Miller media and grown up to OD₆₀₀=0.1. The culture was diluted again 1/500 in 1 ml of fresh media with presence of increasing concentrations of tested compound or 1% DMSO as a control. To calculate CFU the 20 μl aliquots collected in time scale mixed with 180 μl of saline solution and 100 μl volume of 10× serial dilutions, plated on LB Lenox media Petri dish with 1.5% agar and incubated overnight.

Inhibition of bacterial growth. A 97% inhibition of the growth of B. subtilis was observed in the presence of 5 μM RR-THC. No inhibition was seen in the case of Pseudomonas aeruginosa and E. coli strains.

### Example 6: Obtaining an Estimate of MIC from Bacterial Growth Experiments

Drug susceptibility assays. Experiments to determine the minimum inhibitory concentration (MIC) of RR-THC for bacterial growth were performed in 96-well microtiter plates. A DMSO stock solution of each compound was added to the first column and serially diluted across the columns of the plate. The last column of the plate contained no drug and served as a no-drug control. Overnight cultures of the bacterium being tested (MRSA ATCC 43300, MRSA ATCC 25923, or the aforementioned VRSA/VISA strains) were diluted 1000-fold, and 100 μL of the diluted culture (appr. 2×10³ cells) was used as an inoculum in each well. MICs were determined by visual inspection looking for a pellet after 18 h incubation at 37° C. and centrifugation.

Cytotoxicity assay. Compounds were tested for cytotoxicity against mammalian Vero cells using the CellTiter 96 AQueous One Solution Assay kit (Promega). Vero cells were seeded in 96-well plates at a density of 2×10⁴ cells per well, and the plates were incubated for 4 h at 37° C. to allow attachments of the Vero cells. Chemical compounds being tested were then added to the wells starting from a final concentration of 50 μg/mL and making 12 1:2 dilutions. Cells were incubated for 72 h at 37° C., after which 20 μL of freshly prepared MTS:PMS reagents was added to each well. The plates were incubated for 2 h and then read at an absorbance of 490 nm.

### Example 7: Effects of Topical Application of RR-THC on Wound Healing in Mice

### Materials and Methods

Six-week-old female C57Bl/6J mice were purchased from Jackson Laboratories.

Bacterial strain used was USA300 methicillin-resistant Staphylococcus aureus (MRSA) (Lancet 2006 Mar. 4; 367 (9512):731-9).

The chemical compound tested was RR-THC, 20 mg/ml applied as an ointment.

Mupirocin 20 mg/ml (Glenmark Pharmaceuticals) was used as a positive control.

### Testing Protocol:

Testing was done on three groups of mice:
Treated with 20 mg/ml RR-THC—eight animals
Ointment control (no RR-THC added)—eight animals
Positive control, mupirocin 20 mg/ml—eight animals
   Inoculation with Bacteria:

Mice were infected subcutaneously with 2×10⁷ cfu of S. aureus to the shaved backs of mice while under anesthesia (ketamine/xylazine). Five minutes after inoculation, 10 mg of ointment (0.2 mg of drugs) was applied to the regions of inoculation. Ointment was applied daily. Regions of dermonecrosis were measured daily. The experiment was stopped after day 4, 24 h prior to euthanasia and processing. Mice were euthanized through carbon dioxide and confirmed through cervical dislocation. 5 mm punch biopsies were used in the dermonecrotic lesion and homogenized to determine bacterial burden through serial dilution on chromogenic media.

1: A method for inhibiting bacterial protein synthesis in gram-positive bacteria, said method comprising contacting the gram-positive bacteria with a compound comprising Formula I:

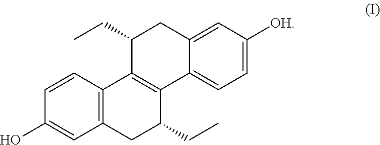 [SMILES: CC[C@@H]1Cc2cc(O)ccc2C2=C1c1ccc(O)cc1C[C@H]2CC]

2: The method of claim 1 wherein the gram-positive bacteria is contacted with the compound in an amount which inhibits ternary complex formation between elongation factor Tu (EF-tu), transfer ribonucleic acid (tRNA) and guanosine triphosphate (GTP) in the gram-positive bacteria.

3: A method for treating a gram-positive bacterial infection in a subject, said method comprising administering to the subject a composition comprising a compound of Formula I:

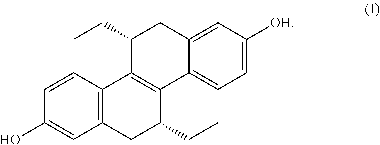 [SMILES: CC[C@@H]1Cc2cc(O)ccc2C2=C1c1ccc(O)cc1C[C@H]2CC]

4: The method of claim 3 wherein the composition is administered to the subject in an amount effective to inhibit ternary complex formation between elongation factor Tu (EF-tu), transfer ribonucleic acid (tRNA) and guanosine triphosphate (GTP) in the gram-positive bacteria.

5: The method of claim 3 wherein the composition is administered orally or topically.

6: The method of claim 4 wherein the composition is administered orally or topically.


SEQUENCE LISTING (2 sequences)

SEQ ID NO: 1 - DNA, 15 bp, Synthetic construct
  source 1..15: mol_type = other DNA
ctctgggaacatcct

SEQ ID NO: 2 - DNA, 20 bp, Synthetic construct
  source 1..20: mol_type = other DNA
tttttaggatgttcccagag